(19) United States
(12) Patent Application Publication
(10) Pub. No.: US 2010/0000948 A1
(43) Pub. Date: Jan. 7, 2010

(54) WATER TREATMENT EQUIPMENT USING PULSED ULTRAVIOLET LAMP

(75) Inventors: Soon Ho Park, Paju (KR); Dae Je Chung, Seoul (KR); Dong Jin Yang, Seoul (KR)

Correspondence Address: VOLPE AND KOENIG, P.C., UNITED PLAZA, SUITE 1600, 30 SOUTH 17TH STREET, PHILADELPHIA, PA, 19103, US

(73) Assignee: GREEN ENVIRONMENTAL TECHNOLOGY CO., LTD., Seoul (KR)

(21) Appl. No.: 12/298,245

(22) PCT Filed: Dec. 27, 2006
(86) PCT No.: PCT/KR2006/005731

§ 371 Date: Jun. 10, 2009

(30) Foreign Application Priority Data
Nov. 24, 2006 (KR) 10-2006-0117038

(51) Int. Cl. C02F 1/32

(52) U.S. Cl. 210/748; 210/198.1; 250/492.1

(57) ABSTRACT

Disclosed therein is a water treatment equipment using pulsed ultraviolet lamp, which emits pulsed ultraviolet rays of high power and high transmission. The water treatment system can be usefully used in a purification plant or in a waste water treatment plant of high turbidity since the UV lamp adapted for emitting the pulsed UV rays is higher in power and longer in a transmission length than the conventional continuous UV lamp.

### TECHNICAL FIELD

The present invention relates to a water treatment equipment using pulsed ultraviolet lamp, and more particularly, to a water treatment equipment using pulsed ultraviolet lamp, which emits pulsed ultraviolet rays of high power and high transmission.

### BACKGROUND ART

Processes for sterilizing various pathogenic bacterium and pathogenic Protozoa including colon bacilli contained in drinking water using ultraviolet (UV) rays are being developed. U.S. Pat. No. 6,565,803 discloses a method for the inactivation of cryptosporidium parvum, such as cryptosporidium oocysts and Giardia cysts, contained in drinking water using UV rays by Bolton et al. Such a sterilizing method using UV rays has an advantage in that it does not generate by-products, such as a carcinogenic substance, differently from the existing chlorination. In addition, as a part of chemical treatment technologies for treating non-biodegradable matters, an oxidation process, in which hydrogen peroxide and UV rays are combined with each other, is being developed.

For such a method for treating water or waste water using UV, there are a water treatment method using a low-pressure mercury lamp and a water treatment method using a medium-pressure mercury lamp. The water treatment technology using the low-pressure mercury lamp uses a lamp, which is filled with mercury gas at a low pressure ranging from 0.007 torr (standard lamp) to 0.76 torr (low pressure and high power) and emits light of 254 mm to about 40%, and is called a standard lamp since it is one of lamps generally used in industries. In general, the mercury lamp has several problems in that it is restricted in commercialization since it is for home use or for experimental use due to a low power ranging from 15 W to 60 W, and in that it is difficult to apply in treating high-density matters and non-biodegradable matters, for instance, waste water. So, as a result of study and development by many lamp manufacturers and lamp utilizing companies, power of the mercury lamp is gradually increased, and so, now, a low pressure and high power lamp of more than 120 W is generally used for sterilization, and a lamp, which can emit light up to 320 W, has been disclosed. A length of the lamp ranges from 1.2 m to 2 m. However, the high power lamp has a problem in that it needs additional cooling device since its surface temperature ranges from 120° C. to 200° C.

The medium-pressure mercury lamp is also filled with mercury gas, but can emit light of several hundreds to several thousands, which is still higher in power than the low-pressure mercury lamp, since pressure of the filling gas is more than 300 torr. However, the medium-pressure mercury lamp has a problem in that the entire UVC generation efficiency including a sterilization wavelength is less than 15% since the UVC generation efficiency is considerably decreased due to an UV resorption phenomenon of mercury gas. In addition, the medium-pressure mercury lamp has additional problems in that the cost of equipment is increased since it needs additional equipment for supplying a great deal of cooling water to cool the lamp due to its surface temperature ranging from 600° C. to 800° C., and in that the cost of maintenance is also increased since the lifespan of the lamp is short.

Furthermore, the medium-pressure mercury lamp is restricted in commercialization since its transmission length is short even though it has a higher power than the low-pressure mercury lamp. The reason is as follows. Since the level that UV transmits waste water is in proportion to an power of the UV lamp but it is difficult for the medium-pressure mercury lamp to secure a transmission distance of more than 10 cm, when a number of the lamps are used to a waste water treatment system of large quantity, it needs immense installation expenses and the number of the cooling devices, which are mounted on the respective lamps, is also increased.

Therefore, there is a need for a new system, which can provide still higher power than the medium-pressure mercury lamp and require no cooling device.

### DISCLOSURE OF INVENTION

### Technical Problem

Accordingly, the present invention has been made in an effort to solve the above-mentioned problems occurring in the prior arts, and it is an object of the present invention to provide a water treatment equipment using a pulsed ultraviolet lamp.

Another object of the present invention is to provide a method for sterilizing water using pulsed ultraviolet rays.

Yet another object of the present invention is to provide a system for sterilizing water using a pulsed ultraviolet lamp.

A further object of the present invention is to provide a method for treating waste water using pulsed ultraviolet rays.

Still further object of the present invention is to provide a system for treating waste water using a pulsed ultraviolet lamp.

### Technical Solution

To achieve the above objects, the present invention provides a water treatment method using pulsed ultraviolet (UV) rays.

In the present invention, the pulsed UV rays are generated in such a way as to apply pulse waveform power source to an UV lamp. The pulse waveform power source can be generated using one of methods well-known to those skilled in the art. In a preferred embodiment of the present invention, after power source supplied by a DC power supplier is accumulated in an electric condenser, current of high energy accumulated in the electric condenser is converted and generated into pulse waveform power source by a pulse generator, and then, the pulse waveform power source is applied to the UV lamp to thereby generate pulsed UV rays. In another preferred embodiment of the present invention, the pulse waveform power source can be generated in such a way as to operate a power supply, which can generate pulse waveform power source, by connecting the power supply to the UV lamp. The power supply, which can generate pulse waveform power source, can be manufactured through the above-mentioned method.

In the present invention, in case where the pulse waveform power source is applied to the UV lamp, since high voltage can generate higher power than low voltage, it is preferable to raise voltage within a range that the lamp can endure. In the embodiment of the present invention, it is preferable that the voltage ranges from 1,000V to 10,000V so as to supply sufficient power. If power source is low, it is impossible to obtain sufficient power due to a low power, but if power source is too high, it is resulted in overload of the lamp. In a preferred embodiment of the present invention, it is preferable that the voltage is more than 1,000V, more preferably, more than 1,500V.

The range of voltage is not restricted theoretically, but if power of the lamp rises, UV rays of short wavelength are generated in large quantities, and it makes decomposition of peroxide and/or waste water easy and may be favorable to a DNA damage of microorganisms.

In the present invention, in case where the pulse waveform power source is applied to the UV lamp, since high power can be generated due to high current, it is preferable to use instantaneous peak current as great as the power supply can generate. In addition, it is preferable to use current ranging from 500 A to 3,000 A, so that power sufficient for sterilization can be generated from the generated UV to thereby increase an amount of useful UVC. In another preferred embodiment of the present invention, it is preferable to use instantaneous peak current of more than 500 A.

In the pulse waveform power source according to the present invention, if a pulse width is narrow, a peak value of pulse gets higher in the same energy and efficiency of UV is increased, so that it serves to raise distribution of UVC. It is preferable that the pulse width ranges from 20 μs to 350 μs, so that power sufficient for sterilization can be generated from the generated UV to thereby increase an amount of useful UVC. If the pulse width is too wide, efficiency lowers, but if too narrow, power is reduced. So, it is preferable to keep the pulse width within the given range.

In the pulse waveform power source according to the present invention, frequency of the pulse can be adjusted in consideration of an environment where the UV lamp is used, but it is preferable to use the pulse within a range that temperature of the lamp can naturally fall. It is preferable that frequency of the pulse is about twelve times per second.

In the present invention, it is preferable that the UV lamp is made of a material having good transmission efficiency, preferably, quartz or sapphire, and can be molded in various forms according to used environments. The UV lamp is filled with inactive gas to prevent resorption of the generated UV and generate a great quantity of UV rays. It is preferable that the inactive gas is xenon or krypton. Xenon or krypton filling the UV lamp can raise filling pressure since it does not cause a resorption phenomenon even though a volume of filling gas is increased. However, to provide the same UV efficiency in case where filling pressure is high, a power supply of higher power is needed. So, it is preferable to use pressure ranging from 100 torr to 1,000 torr.

In an aspect of the present invention, the water treatment is sterilization of water. In the present invention, water may be clean water, sewage or seawater.

The sterilization of water is to sterilize microorganisms, such as Protozoa, bacterium, and animal and plant planktons, living in clean water, sewage and seawater. It is not restricted theoretically, but the sterilization is achieved in such a way as to damage DNA of the microorganisms by emitted UV rays.

The sterilization is achieved through a sterilizing system, on which a lamp adapted for emitting pulsed UV rays is mounted. The sterilizing system includes an UV reactor having an UV lamp mounted thereon. Pulse power source is applied to the UV lamp to emit pulsed UV rays. The UV reactor includes: at least one UV lamp mounted thereon in consideration of a length to transmit UV rays and a size of the UV reactor; an inflow part for introducing water to be treated; a discharge part for discharging treated water; and a power supply adapted for applying pulse waveform power source to the UV lamp. The sterilizing system according to the present invention can treat water in a batch type, a semibatch type or a continuous type, and it is preferable to use the continuous type treatment method to treat a large quantity of clean water, sewage and seawater.

In another aspect of the present invention, the water treatment is to treat waste water containing non-biodegradable matters.

In the present invention, the treatment of waste water is achieved in such a way as to emit pulsed UV rays to decompose peroxide. The peroxide is not specially restricted if it can be decomposed by UV rays and form radicals to decompose a pollution source by reacting with organic maters. Preferably, the peroxide is hydrogen peroxide.

It is not restricted theoretically, but when UV rays are irradiated to waste water, hydrogen peroxide and proton contained in waste water react with each other in mole to mole, so that an OH radical of two moles is generated. The generated OH radical causes a chain oxidation reaction while reacting with hydrogen peroxide, HCO3—, O2—, and organic matters in series, whereby the organic matters are decomposed.

In the present invention, the water treatment can be achieved in a system for treating waste water in such a way as to generate pulsed UV rays to thereby decompose peroxide.

The waste water treatment system according to the present invention includes an UV reactor having an UV lamp mounted thereon. The UV reactor includes: an inflow part for introducing waste water thereto; a discharge part for discharging treated waste water; and at least one UV lamp mounted thereon in consideration of a transmission length of UV rays and a size of the UV reactor. It is preferable that the UV reactor has one lamp mounted at the center thereof to generally irradiate UV rays.

It is preferable that the waste water treatment system further includes a pH controller for controlling pH of waste water introduced into the UV reactor or pH of the discharged waste water. Chemicals for controlling pH may be combination of acid chemicals and basic chemicals, and it is preferable to control pH using a strong acid and a strong base. Preferably, the chemicals for controlling pH are sulphuric acid and sodium hydroxide.

In the present invention, the waste water treatment system includes a unit for inputting peroxide, which is decomposed by pulsed UV rays, preferably, hydrogen peroxide.

An input amount of hydrogen peroxide may be determined according to density of waste water.

The waste water treatment system according to the present invention can treat waste water in a batch type, a semibatch type or a continuous type, and it is preferable to use the continuous type waste water treatment method to treat a large quantity of waste water.

It is preferable that the waste water treatment system further includes a power supply adapted for applying pulse power source to the UV lamp mounted on the UV reactor.

In addition, it is preferable that the waste water treatment system further includes a controlling part for controlling an inflow amount of waste water introduced into the UV reactor, a discharge amount of waste water discharged from the UV reactor, the UV lamp, the power supply, and amounts of pH and peroxide.

In another embodiment of the present invention, the waste water treatment can be achieved by a method for directly decomposing and removing a pollution source by pulsed UV rays. Peroxide can be used as a medium to decompose the pollution source since it can be easily decomposed by UV, but the UV rays in itself can decompose the pollution source.

### Advantageous Effects

The present invention provides a method for treating water using pulsed UV rays. Since the water treatment method using the pulsed UV rays generates higher power than the conventional method using the medium-pressure mercury lamp and can perform natural cooling during the operation, it does not need additional cooling device. In addition, since the pulsed UV rays of high power provide excellent transmission, it can effectively decompose peroxide contained in waste water of high turbidity and sterilize a great deal of water since has a wide transmission range in clean water, such as drinking water. Finally, according to the water treatment method and system of the present invention, the number of necessary lamps per unit volume of waste water or sewage is reduced, and the cost of equipments for treating water of the same volume is reduced since the cooling device is not needed.

### BRIEF DESCRIPTION OF THE DRAWINGS

FIG. 1 is a configurative diagram of a water treatment equipment using a pulsed ultraviolet (UV) lamp according to the present invention.

FIG. 2 is a configurative diagram of a waste water treatment equipment using a pulsed UV lamp according to the present invention.

FIG. 3 is a detailed diagram of the waste water treatment system of FIG. 2.

### MODE FOR THE INVENTION

Reference will be now made in detail to the preferred embodiment of the present invention with reference to the attached drawings.

Sterilization of Water

FIG. 1 illustrates a system for applying a pulsed ultraviolet (UV) lamp of high power according to the present invention in treatment of water, such as clean water, sewage and seawater. The system shown in FIG. 1 includes an UV reactor 1, an inflow part 5 for introducing water to be treated into the UV reactor 1, and a discharge part 6 for discharging treated water. Moreover, the system further includes a power supply 2 for supplying pulse type power source to the UV lamp, and a controlling part 3 for controlling the above parts. Protozoa, bacterium, and animal and plant planktons are sterilized in the UV reactor by UV rays irradiated to a point 4 where the pulsed UV lamp is mounted. The number of the lamps mounted on one UV reactor can be adjusted according to kinds and characteristics of treatment water, and also the number of the UV reactors, through which the introduced treatment water passes, can be adjusted according to a period of stay. The treatment water passing through the UV reactor is inputted to the next reactor or discharged through the discharge part 6. The power supply 2 for operating the lamp mounted on the reactor may be attached above the reactor or arranged in a separate space. Power source for the lamp is supplied to the lamp via an electric wire through an emission part of the power supply. The form and structure of the reactor is designed to operate the pulsed UV lamp, and so, the number of the lamps and dimension of the reactor may be varied according to an installation location of the reactor or characteristics of treatment water.

### FIRST EMBODIMENT

In the system shown in the drawings, pulse waveform power source, which kept operation voltage of about 2,000V, instantaneous current of about 1,000 A, pulse width of 250 μsec, and twelve times operations per second, was applied to the UV lamp filled with xenon gas. A result of a test for sterilizing colon bacilli and general bacterium by applying the pulse waveform power source to inflow water of a settling reservoir in a purification plant is indicated in the following table 1.

TABLE 1
Result of operating this sterilizing system applied to inflow
water of the settling reservoir in the purification plant

| | Number of colon bacilli | Number of bacterium |
|---|---|---|
| Division | (CFU/100 ml) | (CFU/100 ml) |
| Inflow water | 59 | 518 |
| Discharge water | 0 | 10 |
| Removal rate (%) | More than 99.99% | 98% |

(operation time: 3 sec.)

As a result that this sterilizing system was operated for 3 seconds, as indicated in the above table 1, colon bacilli of more than 99.99% and general bacterium of about 98% were sterilized.

### SECOND EMBODIMENT

A result that pulse waves of the same condition as the first embodiment were applied to discharge water of a sewage treatment plant is indicated in the following table 2.

TABLE 2
Result of operating this sterilizing system applied
to discharge water of the sewage treatment plant

| | Number of colon bacilli |
|---|---|
| Division | (CFU/100 ml) |
| Inflow water | 1430 |
| Discharged water | 30 |
| Removal rate (%) | More than 97.9% |

(operation time: 3 sec.)

As a result that this sterilizing system was operated for 3 seconds, as indicated in the above table 2, colon bacilli of about 97.9% were sterilized.

### THIRD EMBODIMENT

A result that pulse waves of the same condition as the first embodiment were applied to an experiment for sterilizing colon bacilli in seawater is indicated in the following table 3.

TABLE 3
Result of operating this sterilizing system
applied to colon bacilli in seawater

| UV | UV | | |
|---|---|---|---|
| irradiation | irradiation | Number of colon bacilli | |
| distance | period | (CFU/100 ml) | Removal rate (%) |
| 10 cm | 0 sec | 169,000 | 0 |
| 10 cm | 3 sec | 1 | More than 99.99% |
| 10 cm | 5 sec | 0 | More than 99.99% |
| 30 cm | 0 sec | 169,000 | 0 |
| 30 cm | 3 sec | 23 | More than 99.98% |
| 30 cm | 5 sec | 0 | More than 99.99% |

As a result that this sterilizing system was operated, as indicated in the above table 3, colon bacilli in seawater were sterilized in a sterilization rate of more than 99.98% at an irradiation distance of 30 cm for 3 seconds.

### FOURTH EMBODIMENT

The same pulse waves as the first embodiment was applied to samples of Cryptosporidium parvum oocysts, which are known as representative pathogenic protozoa the most tolerant to chlorination. A result of discrimination of living and dying the protozoan using an outcyst test of the protozoan and a result of an infection removal rate using the cell culture are indicated in Table 4 and Table 5.

TABLE 4
Result of discrimination of living and dying Cryptosporidium parvum
oocysts according to whether to outcyst

| UV | UV | Total | | |
|---|---|---|---|---|
| irradiation | irradiation | number of | Number of cysts | |
| distance | period | cysts | outcysted (Live) | Live/Total (%) |
| 10 cm | 0 sec | 980 | 889 | 90.7 |
| 10 cm | 3 sec | 991 | 825 | 83.2 |
| 10 cm | 10 sec | 938 | 128 | 13.6 |

TABLE 5
Result of infection removal rate test using the
cell culture of Cryptosporidium parvum oocysts

| | | Number of cysts having | |
|---|---|---|---|
| UV | UV | infection after | |
| irradiation | irradiation | inoculation of | Infection removal |
| distance | period | host cell (number/ml) | rate (%) |
| 10 cm | 0 sec | 4,300 | 0 |
| 10 cm | 3 sec | 0 | More than 99.99% |
| 10 cm | 10 sec | 0 | More than 99.99% |
| 30 cm | 0 sec | 3,800 | 0 |
| 30 cm | 3 sec | 2 | More than 99.95% |
| 30 cm | 10 sec | 0 | More than 99.99% |

As shown in the above Table 4, as the result of discrimination of living and dying Cryptosporidium parvum oocysts according to whether to outcyst, a ratio of living oocysts was 90.7% at the early stage but was reduced to 13.6% after the oocysts were treated with UV at a distance of 10 cm for 10 seconds.

In addition, as shown in the above Table 5, as a result of the infection removal rate test of Cryptosporidium parvum oocysts, the infection removal rate of Cryptosporidium parvum oocysts was nearly one-hundred percent. Finally, in Table 4, even though oocysts of a relatively high rate lived, the infection removal rate was more than 99.95%. So, as you can see from the above, the pulsed UV sterilizing system according to the present invention can effectively sterilize Cryptosporidium parvum oocysts, which are the representative pathogenic protozoa tolerant to chlorination.

In the meantime, Table 6 indicates a test result of comparing the pulsed UV system with the existing commercialized low-pressure mercury lamp system in relation with in the infection removal rate of Cryptosporidium.

TABLE 6
Comparison of infection removal rate of Cryptosporidium parvum
oocysts by UV lamp system

| | UV irradiation distance | | | |
|---|---|---|---|---|
| | 10 cm | | 30 cm | |
| | Irradiation method | | | |
| | | Low- | | Low- |
| | Pulsed | pressure | Pulsed | pressure |
| | UV | mercury UV | UV | mercury UV |
| Number of cysts | 4,300 | 4,300 | 3,800 | 3,800 |
| before treatment | | | | |
| (number/ml) | | | | |
| Number of cysts | 0 | 2100 | 2 | 3,800 |
| after treatment | | | | |
| (number/ml) | | | | |
| Removal rate (%) | 99.99 | 50-60 | 99.95 | 0 |

(UV irradiation period: 3 sec)

As a test result, the conventional low-pressure mercury lamp system showed an infection removal rate ranging from 50 percent to 60 percent even at an irradiation distance of 10 cm, but infection was almost not removed at the distance of 30 cm. Since the pulsed UV system showed the infection removal rate to more than 99.95% even at the distance of 30 cm, the pulsed UV system was still better in performance and still longer in an effective sterilization distance than the conventional low-pressure mercury lamp system.

Waste Water Treatment of Water

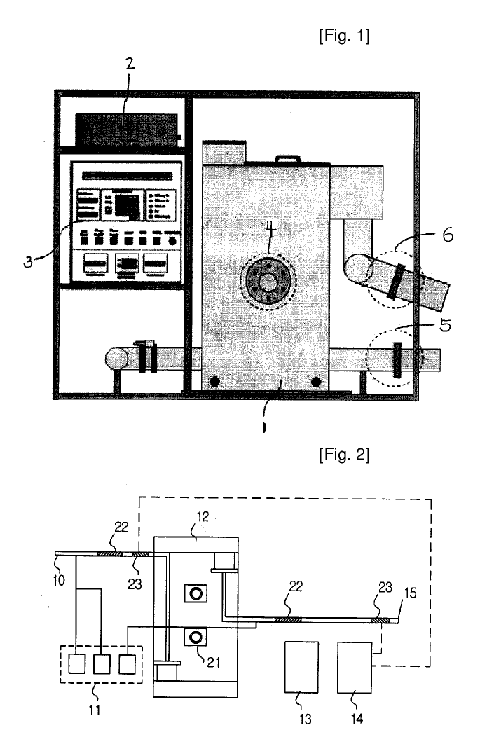

A system shown in FIG. 2 includes an UV reactor 12 having an UV lamp mounted thereon, a waste water inflow part 10 for introducing waste water to the reactor, and a waste water discharge part 15 for discharging waste water. The system further includes: a chemicals input unit 11 for inputting chemicals, for instance, sulphuric acid, sodium hydroxide, and hydrogen peroxide decomposed by UV for decomposing organics, for adjusting pH of the discharged waste water; a power supply 13 for supplying pulsed power source to the UV lamp; and a controlling part 14 for controlling the above parts. A line mixer 22 is mounted on a line to mix the chemicals, and the controlling part controls pH of the line using a pH chip 3 mounted on the line.

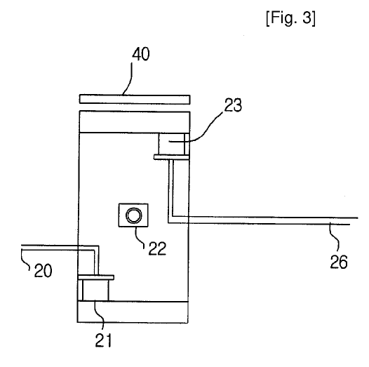

Referring to FIG. 3, the UV reactor will be described in detail. Waste water, which is mixed with the hydrogen peroxide and adjusted in pH to a proper range, is introduced (20), and inputted to the reactor through a baffle 21 of the inflow part mounted on the floor of the reactor. The hydrogen peroxide is decomposed by ultraviolet rays irradiated to a point 22 where the pulsed UV lamp is mounted and generates a great deal of OH radicals to thereby oxidize and remove organics. The number of the lamps mounted on one reactor may be adjust according to kinds and characteristics of waste water, and also the number of the UV reactors, through which the introduced waste water passes, can be adjusted according to a period of stay. The waste water treated through the UV reactor is inputted to the next reactor after passing through an upper baffle 23, or inputted to the next process or discharged through the discharge part 26. The power supply 40 for operating the lamp mounted on the reactor may be attached above the reactor or arranged in a separate space. Power source for the lamp is supplied to the lamp via an electric wire (not shown) through an emission part formed on the rear of the power supply. The form and structure of the reactor is designed to operate the pulsed UV lamp, and so, the number of the lamps and dimension of the reactor may be varied according to an installation location of the reactor or characteristics of treatment water.

### FIFTH EMBODIMENT

In the system shown in the drawings, pulse waveform power source, which kept operation voltage of about 1,800V, instantaneous current of about 1,000 A, operation period of 150 μsec, and ten times operations per second, was applied to the UV lamp filled with xenon gas. In the fifth embodiment, a test result that the water treatment system according to the present invention was applied to waste water discharged from a steel manufacturing company is indicated in the following table 7.

TABLE 7

| Division | COD-Mn(mg/L) | COD-Cr(mg/L) |
|---|---|---|
| Inflow water | 62.2 | 128 |
| Discharge water | 6.1 | 11.2 |
| Removal rate (%) | 90.2 | 91.2 |

As shown in the above Table 7, the removal rate of COD-Mn was about 90.1%, and the removal rate of COD-Cr was about 91.2%.

### SIXTH EMBODIMENT

In the sixth embodiment, the water treatment system according to the present invention was applied, and the test result is indicated in the following Table 8.

TABLE 8

| Division | COD-Cr(mg/L) |
|---|---|
| Inflow water | 141 |
| Discharge water | 19.3 |
| Removal rate (%) | 86.3 |

As shown in the above Table 8, COD-Cr to be removed was removed to about 86.3% by the system.

The above embodiments are just examples of the water treatment system, and so, it would be appreciated that they are not restricted to specific drinking water or waste water.

1. A water treatment method of treating water using an ultraviolet (UV) lamp, which generates pulsed UV rays of high power and high transmission.

2. The water treatment method according to claim 1, wherein the pulsed UV rays are emitted by pulse waveform power source applied to the UV lamp.

3. The water treatment method according to claim 2, wherein the UV lamp is filled with pressure ranging from 100 torr to 1,000 torr.

4. The water treatment method according to claim 2, wherein the pulse waveform power source keeps operation voltage ranging from 1,000V to 10,000V, instantaneous peak current ranging from 500 A to 3,000A, and pulse wave width ranging from 20 [symbol] to 350 [symbol] .

5. The water treatment method according to claim 1, wherein the UV lamp is filled with inactive gas, such as xenon or krypton.

6. A sterilizing system for sterilizing water using an UV lamp, which irradiates pulsed UV rays.

7. The sterilizing system according to claim 6, wherein the pulsed UV rays are emitted in such a way as to apply pulse waveform power source to the UV lamp.

8. The sterilizing system according to claim 7, wherein the UV lamp is filled with pressure ranging from 100 torr to 1,000 torr.

9. The sterilizing system according to claim 7, wherein the pulse waveform power source keeps operation voltage ranging from 1,000V to 10,000V, instantaneous peak current ranging from 500 A to 3,000 A, and pulse wave width ranging from 20 [symbol] to 350 [symbol] .

10. The sterilizing system according to claim 7, wherein the UV lamp is filled with inactive gas, such as xenon or krypton.

11. A waste water treatment system, which emits pulsed UV rays by applying pulse waveform power source to an UV lamp and decomposes peroxide contained in waste water using the pulsed UV rays to thereby treat the waste water.

12. The waste water treatment system according to claim 11, wherein the pulse waveform power source is generated by a pulsed power supply.

13. The waste water treatment system according to claim 11, wherein the pulse waveform power source keeps voltage ranging from 1,000V to 3,000V.

14. The waste water treatment system according to claim 11, wherein the UV lamp is filled with inactive gas.

15. A waste water treatment system comprising a waste water inflow and outflow part, an UV reactor, an UV lamp, a power supply for supplying pulse waveform power source to the UV lamp, a chemicals input unit for adjusting peroxide and pH, and a controlling part, wherein the power supply supplies pulse waveform power source, which keeps voltage ranging from 1,000V to 3,000V and peak current ranging from 1,000 A to 1,500 A.

16. The waste water treatment system according to claim 15, wherein the UV lamp is filled with xenon gas or krypton gas at pressure ranging from 500 torr to 1,000 torr.

17. The waste water treatment system according to claim 15, wherein the peroxide is hydrogen peroxide.
